(19) United States
(12) Patent Application Publication
(10) Pub. No.: US 2020/0221931 A1
(43) Pub. Date: Jul. 16, 2020

(54) ENDOSCOPIC STABILIZING TOOLS AND RELATED METHODS OF USE

(71) Applicant: Boston Scientific Scimed, Inc., Maple Grove, MN (US)

(72) Inventors: Evan Wilder, Boston, MA (US); Scott E. Brechbiel, Acton, MA (US); James Weldon, Newton, MA (US)

(73) Assignee: Boston Scientific Scimed, Inc., Maple Grove, MN (US)

(21) Appl. No.: 16/741,904

(22) Filed: Jan. 14, 2020

Related U.S. Application Data
(60) Provisional application No. 62/792,579, filed on Jan. 15, 2019.

(51) Int. Cl. A61B 1/00

(52) U.S. Cl. CPC A61B 1/00098; A61B 1/00137; A61B 1/00158; A61B 1/00082

(57) ABSTRACT

A system includes a member having a lumen; and a stabilizer positioned at a distal end of the lumen, wherein: the stabilizer includes at least two radially-inward projections circumferentially spaced apart from one another at the distal end of the lumen; the at least two radially-inward projections defining an opening at the distal end of the lumen; and a cross-sectional area of the opening is less than a cross-sectional area of the lumen.

### CROSS-REFERENCE TO RELATED APPLICATION(S)

This application claims the benefit of priority from U.S. Provisional Application No. 62/792,579, filed on Jan. 15, 2019, which is incorporated by reference herein in its entirety.

### TECHNICAL FIELD

Various aspects of the present disclosure relate generally to endoscopic devices. More specifically, the present disclosure relates to endoscopic stabilizing and related methods of use.

### BACKGROUND

During both diagnostic and therapeutic endoscopic procedures, accessory devices may be passed through the working channel of the endoscope. The outer diameter of the accessory device should be compatible with the inner diameter of the working channel. Endoscopes used solely for diagnostic procedures generally have smaller working channels, compared to those used for combination (diagnostic and therapeutic) or solely therapeutic procedures. For example, diagnostic and therapeutic gastroscopes typically have working channel inner diameters of 2.8 mm and 3.7 mm, respectively. Accessory devices designed for use in diagnostic scopes are generally compatible with therapeutic scopes as well. However, accessory devices designed for use in diagnostic scopes may be undersized when used with therapeutic scopes, resulting in a loose fit within the working channel.

This loose fit can lead to accessory device instability as the scope articulates throughout the procedure. Accessory device instability during the procedure may result in variable orientation of the device within the working channel, as seen under direct visualization. Though device instability may not be problematic during some procedures, it can be an issue during more precise procedures (such as, e.g., endoluminal surgery). During endoluminal surgical procedures, a cutting knife may be used to excise tissue. Some existing cutting knives have no articulation capability, and cutting motions performed by the physician are controlled by articulation of the scope. In cases where the cutting knife is undersized relative to the inner diameter of the working channel of the endoscope, there is a loose fit between the knife and the working channel, and thus, the knife may move unexpectedly as the physician articulates the scope. This introduces a level of unpredictability for the physician performing the procedure and potential risk for the patient.

### SUMMARY

A system includes a member having a lumen; and a stabilizer positioned at a distal end of the lumen, wherein: the stabilizer includes at least two radially-inward projections circumferentially spaced apart from one another at the distal end of the lumen; the at least two radially-inward projections defining an opening at the distal end of the lumen; and a cross-sectional area of the opening is less than a cross-sectional area of the lumen.

Each of the at least two projections is a ramp that extends from a proximal end toward a distal end, and has an increasing radial dimension extending from the proximal end toward the distal end. The stabilizer includes a ring-shaped support secured within the lumen, and each of the ramps extends into the lumen from the ring-shaped support. The stabilizer includes a cap configured to extend over a distal end of the scope. The stabilizer includes a first ring positioned within the lumen, and a second ring disposed within the first ring, and rotatable relative to the first ring; and the ramps extend into the lumen from the second ring. The system includes a working tool insertable into the lumen. The first ring includes a circumferential flange; and the ramps are configured to urge the working tool against the circumferential flange. Rotation of the working tool in a first direction causes the working tool to rotate along the circumferential flange and around the lumen in a second direction that is opposite of the first direction. Rotation of the working tool in the first direction also causes the second ring to rotate in the second direction. When the first direction is clockwise, the second direction is counter-clockwise; and when the first direction is counter-clockwise, the second direction is clockwise. The ramps are configured to rotate about a central longitudinal axis of the lumen. The stabilizing tool includes one or more gears configured to rotate the ramps. The system includes a twistable member extending from a proximal end of the member to one of the gears, wherein rotation of the twistable member is configured to rotate each of the gears and the ramps. A free end of each of the radially-inward projections is configured to flex distally away from a distal end of the member. The system includes a working tool insertable into the lumen, wherein: the radially-inward projections extend into the lumen from a first side of the lumen; are configured to be flexed distally by the working tool; and bias the working tool toward a second side of the lumen that is across a central longitudinal axis of the lumen from the first side.

A system includes a member having a lumen; and a flexible stabilizer fixed to a distal end of the lumen, wherein: the flexible stabilizer is movable from a collapsed position to an expanded position extending distally away from the distal end of the lumen; the flexible stabilizer includes a central longitudinal axis; and the flexible stabilizer is biased toward the collapsed position and toward a central longitudinal axis of the flexible stabilizer, to define an instrument-containing space having a cross-sectional dimension less than a diameter of the lumen.

The flexible stabilizer is a coil, spring, or ribbon, and the lumen includes a central longitudinal axis that is offset from the central longitudinal axis of the coil, the spring, or the ribbon.

A system includes a member having a lumen; and a first sleeve dimensioned to be received in the lumen; and a first magnet disposed within or adjacent to a distal end of the lumen, wherein: a distal end of the first sleeve includes a second magnet or a ferromagnetic material; a proximal end of the first sleeve is non-magnetic; and the first sleeve includes a lumen configured to receive a working tool.

The first magnet is a ring surrounding the lumen. The second magnet or the ferromagnetic material of the first sleeve extends only partially around a circumference of the first sleeve, and rotation of the first sleeve in a first direction causes the first sleeve to rotate around the lumen in the first direction.

### BRIEF DESCRIPTION OF THE DRAWINGS

The accompanying drawings, which are incorporated in and constitute a part of this specification, illustrate exemplary aspects of the present disclosure and together with the description, serve to explain the principles of the disclosure.

FIG. 1 is a perspective view of an endoscope and a stabilizing tool.

FIG. 2 is a side view of the stabilizing tool of FIG. 1, with internal components shown through the outer surface of an endoscope working channel.

FIGS. 3-6 are side views of an endoscope and stabilizing tool, according to another embodiment, with internal components shown through the outer surface of the endoscope.

FIG. 7 is a front view of the distal end of the endoscope and stabilizing tool of FIGS. 3-6.

FIGS. 8-11 are side views of an endoscope and a stabilizing tool, with internal components shown through the outer surface of the endoscope, according to yet another embodiment.

FIG. 12 is a front view of the distal end of the endoscope, according to yet another embodiment.

FIGS. 13 and 14 are perspective views of an endoscope and a stabilizing tool, according to yet another embodiment.

FIGS. 15 and 16 are perspective views of an endoscope and a stabilizing tool, according to yet another embodiment.

FIG. 17 is a perspective cross-sectional view of the endoscope and stabilizing tool of FIGS. 15 and 16.

FIG. 18 is a perspective view of an endoscope and a stabilizing tool, according to yet another embodiment of the present disclosure.

FIG. 19 is a perspective view of an endoscope and a stabilizing tool, according to yet another embodiment of the present disclosure.

FIG. 20 is a perspective view of an endoscope and a stabilizing tool, with internal components shown through the outer surface of the endoscope, according to yet another embodiment.

FIG. 21 is a perspective view of an endoscope and a stabilizing tool, according to yet another embodiment.

FIG. 22 is a schematic view of a portion of the stabilizing tool of FIG. 21.

### DETAILED DESCRIPTION

Reference will now be made in detail to aspects of the present disclosure, examples of which are illustrated in the accompanying drawings. Wherever possible, the same or similar reference numbers will be used through the drawings to refer to the same or like parts. The term “distal” refers to a portion farthest away from a user when introducing a device into a patient. By contrast, the term “proximal” refers to a portion closest to the user when placing the device into the patient. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” Further, relative terms such as, for example, “about,” “substantially,” “approximately,” etc., are used to indicate a possible variation of ±10% in a stated numeric value or range.

Embodiments of this disclosure seek to improve stability for accessory devices (e.g., working tools) within a lumen or working channel of a scope, such as, e.g., an endoscope, particularly for tools that are undersized relative to the lumen. Similar to an elevator component in a duodenoscope, a steerable component may be included in some embodiments, to give a user an additional degree of freedom when manipulating the tools at a distal end of the scope.

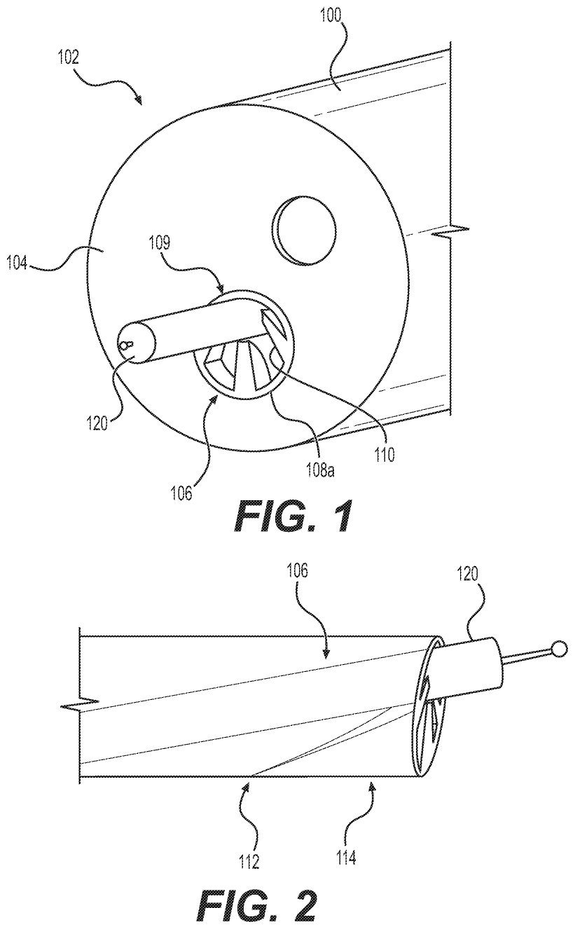

FIGS. 1 and 2 show a scope 100 that extends from a proximal end (not shown) to a distal end 102. Distal end 102 may include a distally-facing surface 104 having a lumen 106 (e.g., a working channel). Scope 100 may be any suitable endoscopic member, such as, e.g., an endoscope, a ureteroscope, a nephroscope, a colonoscope, a hysteroscope, a uteroscope, a bronchoscope, a cystoscope, a duodenoscope, a sheath, or a catheter. Scope 100 may include one or more additional lumens configured for the passage of a variety of therapeutic or diagnostic equipment, including, but not limited to, imaging devices and tools for irrigation, vacuum suctioning, biopsies, and drug delivery. It is also contemplated that distally-facing surface 104 may include an imaging device (e.g., camera) embedded, or otherwise fixed, therein. At least a portion of scope 100 may be radiopaque.

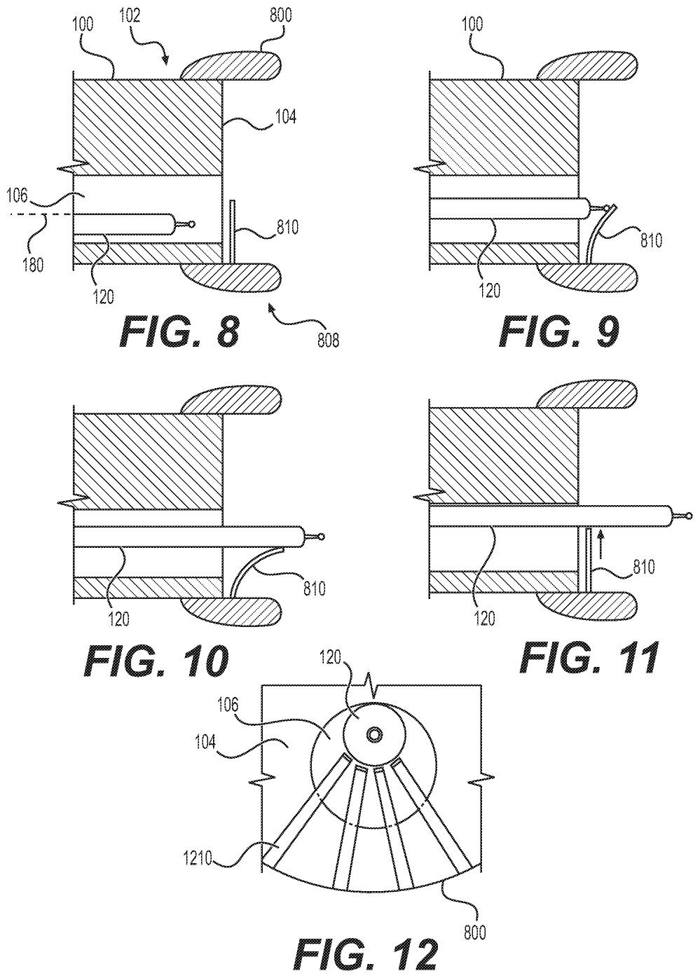

A stabilizer, or stabilizing tool 108a may be disposed in a distal end of lumen 106. Stabilizing tool 108a may be configured to stabilize and guide a working tool 120 movable through lumen 106. Stabilizing tool 108a may include a ring-shaped support that may be removably secured within the distal end of lumen 106, or the ring-shaped support may be fixed with a body of scope 100. When stabilizing tool 108a is removable relative to lumen 106, stabilizing tool 108a may be secured within lumen 106 by a friction or interference fit, or another suitable fit. In another embodiment, stabilizing tool 108a may be provided in a cap configured to surround distal end 102 of scope 100 (see e.g., cap 800 described below with respect to FIGS. 8-11). Stabilizing tool 108a may be configured to accommodate and stabilize a single (exactly one) working tool 120 at a time by reducing an effective diameter of lumen 106 at distal end 102. For example, stabilizing tool 108a may create a reduced-diameter opening 109 at the distal end of lumen 106. The cross-sectional area of opening 109 may be less than a cross-sectional area of lumen 106. The cross-sectional area of opening 109 may be, e.g., from about 5% to about 95% of the cross-sectional area of lumen 106.

Stabilizing tool 108a may include one or more ramps 110 that extend, from an inner circumferential surface of stabilizing tool 108a, radially into lumen 106. In some embodiments, stabilizing tool 108a may include at least two ramps 110. The embodiment in FIG. 1 includes three ramps 110. Ramps 110 may be circumferentially spaced from one another, and each ramp 110 may project toward a center of lumen 106 and opening 109. Ramps 110 may extend from a proximal end 112 toward a distal end 114, and may have an increasing radial dimension moving from proximal end 112 toward distal end 114. In other words, ramps 110 taper in a radial dimension toward proximal end 112. Ramps 110 may help guide a given working tool 120 distally through lumen 106 to the reduced-diameter opening 109. Ramps 110 may extend radially into lumen 106 by a largest amount at distal end 114 (as seen in FIG. 2). Furthermore, distal ends 114 of ramps 110 may be disposed at a distalmost portion of lumen 106, or slightly proximal to the distalmost portion of lumen 106. While multiple ramps 110 are shown in FIG. 1, stabilizing tool 108a alternatively may have a single ramp that extends partially around a circumference of lumen 106 (e.g., between 5 and 355 degrees around lumen 106). This range is only exemplary, and other suitable ranges also are contemplated. The ratio of the total cross-sectional area of distal ends 114 relative to the cross-sectional area of lumen 106 may be less than about 0.5, less than about 0.4, less than about 0.3, less than about 0.2, or less than about 0.1. Stated differently, the total cross-sectional area of ramps 110 may cover only a minority of the total cross-sectional area of lumen 106. However, it also is contemplated in some embodiments, that the total cross-sectional area of distal ends 114 may cover a majority of the cross-sectional area of lumen 106 (e.g., a ratio greater than 0.5). Ramps 110 may be rigid, or may have some flexibility.

Components of stabilizing tool 108a may be fixed relative to one another, and thus, stabilizing tool 108a may be configured to accommodate and stabilize tools 120 within a small range of diameters (e.g., tools having only one diameter). For example, if a stabilizing tool 108a has a reduced-diameter opening 109 with a diameter of 2 mm, then that stabilizing tool 108a may be configured to receive only tools 120 with an approximate diameter of 2 mm (while stabilizing tool 108a in this example may receive tools 120 having a smaller diameter, such an arrangement may result in less stabilization). For working tools 120 having larger diameters, e.g., 3 mm or 5 mm diameters, two different stabilizing tools 108a (having respective diameters of 3 mm and 5 mm) may be required. In some embodiments, stabilizing tool 108a may be removed from lumen 106 by applying a distally-directed force to proximal end 112 of ramp 110, or to another portion of stabilizing tool 108a. In one embodiment, stabilizing tool 108a may include a flat and proximally-facing surface against which the distally-directed force may be applied.

One or more portions of stabilizing tool 108a may include a tacky coating to help secure working tool 120 at distal end 102. In some embodiments, distal portions of stabilizing tool 108a may include the tacky coating while proximal portions of stabilizing tool 108a may have no coating or a lubricious coating to allow tool 120 to slide relative to tool 108a, e.g., on ramps 110 of tool 108a. The tacky coating may comprise a tacky silicone, such as, e.g., a moisture cured silicone or polydimethylsiloxane. Other tacky polymeric materials may be used, such as, e.g., styrenic block copolymers (e.g., styrene-isobutylene-styrene (SIBS), styrene-ethylene/butylene-styrene (SEBS), styrene-ethylene/propylene-styrene (SEPS), and styrene-isoprene-styrene (SIS)), acrylics, polyvinyl ether, polyurethanes, and copolymers of ethylene, or the like. For surfaces of tool 108a that have less friction (such as radially inward surfaces ramps 110), various hydrophilic and lubricious coatings may be used.

Working tool 120 may be any tool known to one of skill in the art. For example, the tool may include a grasper, a forceps, a snare, a scissor, a knife, a dissector, a clamp, an endoscopic stapler, a tissue loop, a clip applier, a suture-delivering instrument, or an energy-based tissue coagulator or cutter.

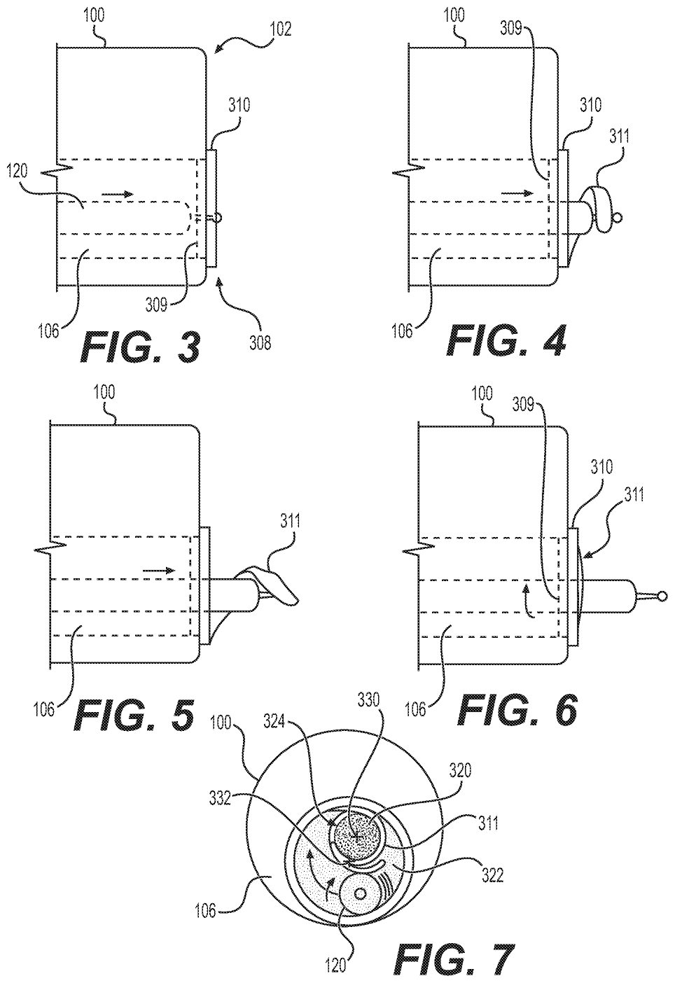

FIGS. 3-7 show endoscope 100 having a stabilizing tool 308 positioned within lumen 306. Stabilizing tool 308 may include an insert 309 insertable into the distal end of lumen 106, or may include a cap configured to positioned over an entirety of distal face 104 of scope 100 (such as, e.g., cap 800, described below with reference to FIGS. 8-11). Stabilizing tool 308 also may include a flange 310 configured to abut against distal face 104, and an expandable member 311 movable from a collapsed position (e.g., FIGS. 3 and 6) to one or more expanded locking configurations (e.g., FIGS. 4 and 5). Insert 309 may have a diameter that is slightly less than the diameter of lumen 106, while flange 310 may have a diameter that is larger than the diameter of lumen 106.

Expandable member 311 may be a coil, spring, or ribbon, and may include stainless steel, nitinol, or a flexible polymer. Expandable member 311 also may include any suitable materials to produce friction and grip between working tool 120 and expandable member 311. For example, the inner radial surfaces of expandable member 311 may include a tacky coating, while the outer radial surfaces of expandable member 311 may include a lubricious coating. Similar to stabilizing tool 108a, stabilizing tool 308 may be configured to accommodate and stabilize working tools 120 of a single, approximate diameter. However, expandable member 311 may have some flexibility due its material thickness, shape, etc., so that stabilizing tool 308 may accommodate working tools 120 of varying size and diameter.

Stabilizing tool 308 may divide lumen 106 into a holding region 320 and a non-holding region 322. Expandable member 311 may radially surround holding region 320, and non-holding region 322 may comprise the remainder of lumen 106 not encompassed by holding region 320. Thus, the combined cross-sectional areas of holding region 320 and non-holding region 322 may be substantially equal to the cross-sectional area of lumen 106. A central longitudinal axis 330 of holding region 320 may be offset from a central longitudinal axis 332 of lumen 106. Holding region 320 may be disposed adjacent to a periphery of lumen 106. In one embodiment, holding region 320 may abut an inner circumferential surface of scope 100 that defines lumen 106. At least a portion of expandable member 311 defining holding region 320 may include a radially-inward directed bias toward central longitudinal axis 330, to help secure a working tool 120 disposed in holding region 320. Expandable member 311 also may be biased toward the collapsed positions of FIGS. 3 and 6.

Working tool 120 may be secured within lumen 106 when positioned within holding region 320. Working tool 120 is not stabilized within lumen 106 (e.g., is loose and unrestricted in lumen 106) when it is advanced into and through non-holding region 322. Referring to FIG. 7, when working tool 120 is disposed within non-holding region 322, rotation of working tool 120 about its axis at its proximal end by the user may move working tool 120 around the circumference of lumen 106. This rotation may be performed until working tool 120 enters holding region 320 via a side-facing opening 324, and locks or is otherwise secured into holding region 320. It should be noted that rotation in only one of two directions will result in working tool 120 moving into holding region 320. The lubricious coating on the outer radial surfaces of expandable member 311 (and/or the surface defining lumen 106) may assist with this rotation.

In use, working tool 120 may be inserted into a proximal portion of lumen 106, through lumen 106, and then used to push expandable member 311 distally away from distal end 102 of scope 100. In particular, as working tool 120 extends distally though holding region 320, the inner radial surfaces of expandable member 311 may grip onto the outer surface of working tool 120, causing expandable member 311 to extend distally. In some embodiments, stabilizing tool 308 may retract (e.g., snap back) into the collapsed position, when working tool 120 is retracted from scope 100. In some embodiments, stabilizing tool 308 may be a single-use device. For example, after working tool 120 is passed through holding region 320 and expandable member 311 is extended distally, the deformation experienced by expandable member 311 may prevent subsequent uses. However, in other embodiments, stabilizing tool 308 may be suitable for multiple uses.

Referring to FIGS. 8-12, endoscope 100 is shown with a stabilizing tool 808 that is configured to accommodate and stabilize working tools 120 of differing diameters. Stabilizing tool 808 may include a cap 800 configured to attach to distal end 102 of scope 100 by, e.g., a friction or interference fit. A flexible member 810 may extend radially inward from an inner circumferential surface of cap 800. When cap 800 is installed on scope 100, flexible member 810 may extend from one side of lumen 106, toward (and in some embodiments, across) a central longitudinal axis 180 of lumen 106, to an opposing side of lumen 106. Flexible member 810 may be configured to flex or bend in one or more directions. For example, as shown in FIGS. 8-11, flexible member 810 may be pushed distally by working tool 120. The flexed condition of flexible member 810, and its bias to the state shown in FIG. 8, may urge working tool 120 toward the opposing side of lumen 106, while securing working tool 120 on the opposing side. Based on the diameter of working tool 120, the flexed position of flexible member 810 in a locking configuration may differ. For example, when working tool 120 has a first diameter (e.g., as shown in FIGS. 8-11), flexible member 810 may show relatively little flexion in the locking configuration. However, when working tool 120 has a larger diameter than shown in FIGS. 8-11, flexible member 810 may exhibit more flexion when in the locking configuration. While a single flexible member 810 is shown in the embodiment of FIGS. 8-11, as shown in FIG. 12, multiple flexible members 1210 alternatively may be utilized. The multiple flexible members 1210 may be circumferentially spaced about cap 800 and lumen 106. The embodiment shown in FIG. 12 may allow additional suction through lumen 106 due to less of its distal opening being covered.

Embodiments of the present disclosure may function as displacement tools to securely position working tool 120 within lumen 106 of scope 100. Secure positioning of working tool 120 by the various stabilizing tools set forth above may allow for increased user control for working tools 120 in oversized lumens 106. Though working tool 120 may be precisely positioned at distal end 102, the stabilizing tools may allow working tool 120 to move freely within lumen 106 at the proximal ends of scope 100 and lumen 106. The stabilizing tools could be single-use or integrated into scope 100. In the case of the single-use application, the stabilizing tools could include a cap inserted over distal end 102 of scope 100 (e.g., cap 800), or the stabilizing tool may be inserted directly into lumen 106. The stabilizing tools may position tool 120 in a variety of different locations within the lumen 106 (e.g., the center of lumen or along its edge). The selected positioning may depend on a user's preferred visualization and placement of working tool 120 at distal end 102. The stabilizing tools may position tool 120 via discrete contact points or continuous circumferential contact. In some embodiments, a long circumferential member may obscure the suction capability of scope 100, and thus discrete contact points may be advantageous in applications where lumen 106 is used for fluid suctioning or fluid delivery. The size and position of the contact points may be optimized to achieve desired positioning of tool 120, and suction capability.

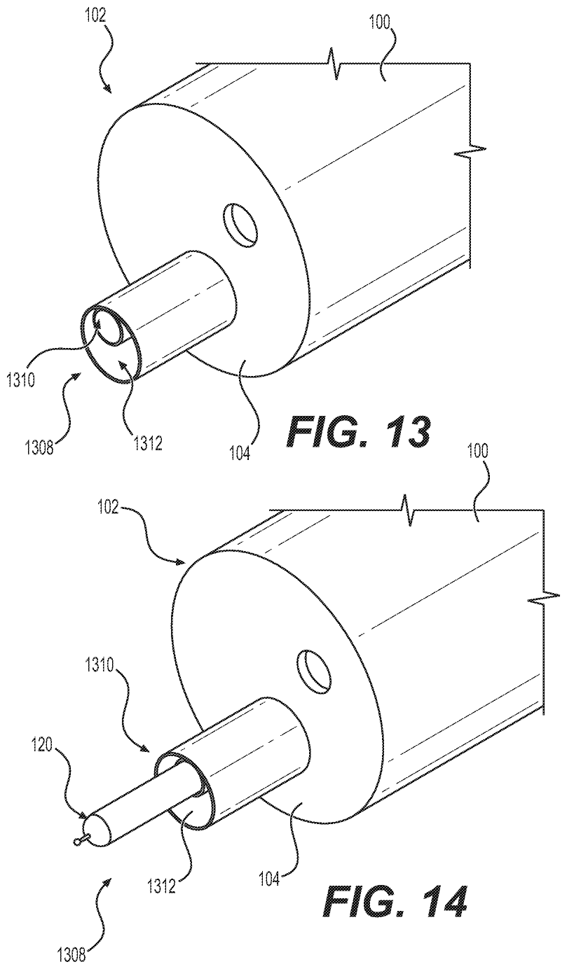

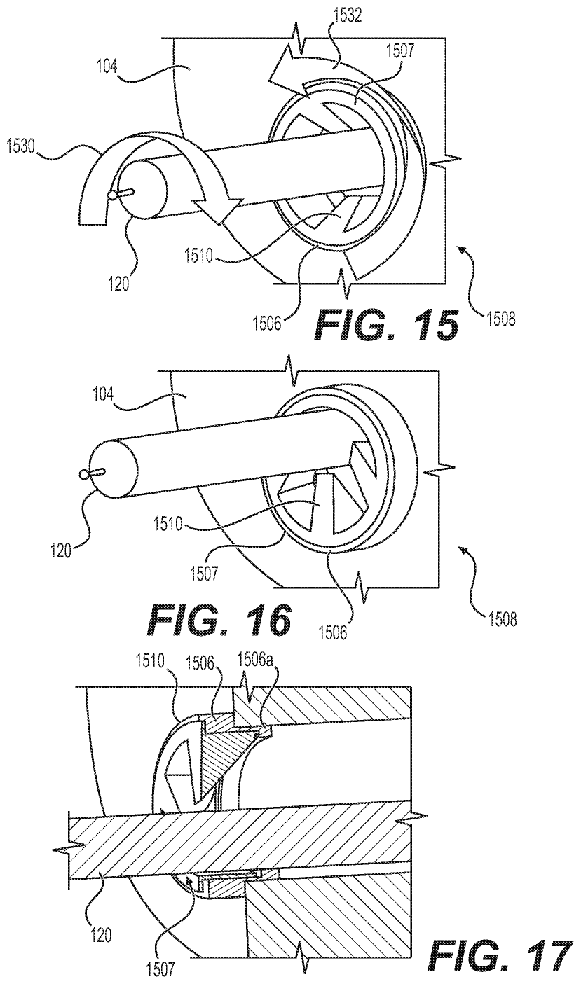

In the embodiment shown in FIGS. 13 and 14, a stabilizing tool 1308 may separate lumen 106 into a tool channel 1310 and a suction channel 1312, creating a parallel-tube (dual-tube) configuration. Tool channel 1310 may be disposed within suction channel 1312, and may be fixed relative to suction channel 1312. Tool channel 1310 may help provide stability for working tool 120, and separate working tool 120 from the dedicated suction channel 1312. Tool channel 1310 may have a slightly larger diameter than working tool 120, to allow working tool 120 to slide through channel 1310, but otherwise remain secure within the channel, Suction channel 1312 may extend through the length of scope 100, and may be coupled to an external waste container (not shown) that is exterior of scope 100. Tool channel 1310 may extend through lumen 106 to a biopsy port at a handle (not shown) of scope 100. The dual-tube stabilizing tool 1308 itself also may be movable within lumen 106. In some embodiments, suction could be applied without placing distal end 102 of scope 100 directly in the liquid to be suctioned. For example, to suction a fluid located distal of distal end 102, an entirety of stabilizing tool 1308 may be extended distally from distal end 102 into to the liquid so that distal end 102 is spaced apart from the liquid. This may enable a user to maintain visualization while applying suction, since the imaging tools of scope 100 may not be submerged in liquid during the suction procedure.

Stabilizing tools of the present disclosure may be either passive (e.g., not requiring any additional user intervention to stabilize a working tool other than the insertion of working tool 120) or active (e.g., requiring or permitting additional user action to secure working tool 120 or providing additional operator control—examples of which are described below). Active displacement tools may include ON/OFF and/or direction control. Direction control for the active displacement tools may allow users, e.g., physicians or other medical practitioners, to steer the tool 120 within lumen 106 of scope 100 at distal end 102, similar to the elevator action in a duodenoscope. Direction control for the active displacement tools may be achieved via mechanical power (e.g., twisting of working tool 120 or a knob by the user) or electrical power (e.g., servomotor).

FIGS. 15-17 show a stabilizing tool 1508, where rotation of tool 120 itself about its own axis, is configured to rotate tool 120 around lumen 106. Stabilizing tool 1508 may include a first ring 1506 and a second ring 1507. First ring 1506 and second ring 1507 may be concentric, and first ring 1506 may surround second ring 1507. First ring 1506 may be secured into lumen 106 by a friction or interference fit, and second ring 1507 may be configured to rotate relative to first ring 1506. For example, first ring 1506 and second ring 1507 may include corresponding features configured to secure first ring 1506 and 1507, and still provide for relative rotation. The complementary features include, but are not limited to, flanges, tracks, recesses, rails, bearings, and the like. Furthermore, the inner surfaces of first ring 1506 and the outer surfaces of second ring 1507 that contact one another may be coated with lubricant and/or may include lubricious coatings.

Second ring 1507 may include similar features as stabilizing tool 108a discussed above. For example, second ring 1507 may include one or more ramps 1510 that are substantially similar to ramps 110 discussed above. Referring to FIG. 17, ramps 1510 may push working tool 120 toward the periphery of lumen 106 and into contact with a circumferential flange 1506a of first ring 1506. Flange 1506a may include a tacky coating or may be formed of rubber or another suitable material that forms a high coefficient of friction with the outer surface of working tool 120. Rotation of working tool 120 itself, about its own central axis, may rotate tool 120 around lumen 106. For example, rotating working tool 120 (in direction 1530) may rotate tool 120 along flange 1506a (e.g., the inner edge) of first ring 1506 in a direction 1532 that is opposite of direction 1530. As working tool 120 moves around lumen 106 in direction 1532, it may push against second ring 1507 riding inside first ring 1506 (specifically against a ramp 1510 of second ring 1507), causing second ring 1507 to rotate in the direction 1532. Material selection of first ring 1506, second ring 1507, and working tool 120 may be important in this embodiment, as second ring 1507 may freely rotate relative to first ring 1506 (low coefficient of friction), while working tool 120 and first ring 1506 (flange 1506a) must not slip (high coefficient of friction). The surface of flange 1506a, where contact is made between flange 1506a and working tool 120, could be coated with an elastomer like neoprene, rubber, silicone, or like materials. First ring 1506 a polymer that the elastomer can adhere to while still holding a stiff shape. Second ring 1507 may include PTFE, or a material having a similar lubricity.

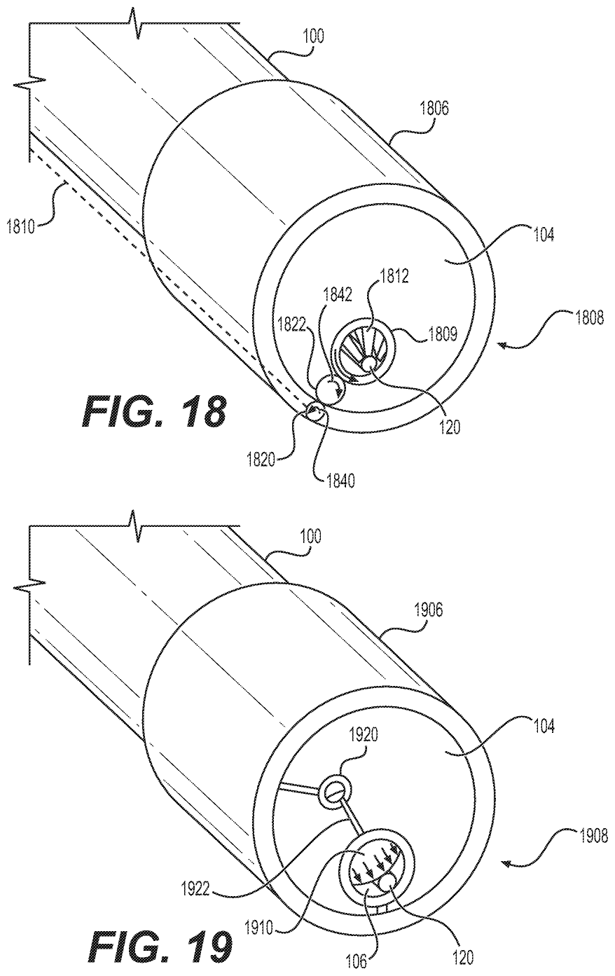

FIG. 18 shows endoscope 100 with a stabilizing tool 1808 having an endoscope cap 1806, a gripping insert 1809, and a twistable member 1810. Gripping insert may include one or more ramps 1812 (having any of the features described in other embodiments with ramps), and may secure working tool 120 in a substantially similar manner as ring 1507 described above. Gripping insert 1809 may include one or more portions that are inserted into lumen 106 of scope 100. Rotation of the twistable member 1810 (about a central longitudinal axis of twistable member 1810) may rotate gripping insert 1809 (and working tool 120 disposed within gripping insert 1809) about lumen 106. Twistable member 1810 may be a cord, wire, cable, or the like extending parallel to the length of scope 100. Twistable member 1810 may be rotated manually by the user (via, e.g., a crank), or automatically (via, e.g., a servomotor). Stabilizing tool 1808 may include one or more gears coupled to cap 1806 to help effectuate rotation of working tool 120. The gears may be positioned distally of distal face 104. For example, portions of the gears may abut distal face 104 when endoscope cap 1806 is positioned on the distal end 102 of scope 100. A first gear 1820 may be directly coupled to a distal end of twistable member 1810. Twisting of member 1810 may cause first gear 1820 to rotate in the first direction 1840. Teeth of first gear 1820 may be configured to interact with teeth of a second gear 1822, causing second gear 1822 to rotate in a second direction 1842 that is opposite of first direction 1840. The teeth of second gear 1822 may be configured to interact with and rotate teeth of gripping insert 1809 (and tool 120 disposed therein) in the first direction 1840. It is contemplated that any suitable number and type of gears may be utilized in any configuration in order to convert a rotational force applied to twistable member 1810 into rotation of gripping insert 1809. For example, in some embodiments, such as, when there is no second gear 1822, or when there is an even number of intermediate gears between first gear 1820 and gripping insert 1809, rotation of twistable member 1810 in the first direction 1840 may cause gripping insert 1809 and tool 120 to rotate in the second direction 1842.

Twistable member 1810 also may be disposed within lumen 106, extending through the biopsy port at a proximal end of scope 100. In this alternative embodiment, rotation of twistable member 1810 about its central longitudinal axis may transfer rotation via first gear 1820 to stabilizing tool 1808. Second gear 1822 may or may not be incorporated due to spacing. The alternative embodiment may require a larger working channel, such as a 6 mm working channel gastroscope.

FIG. 19 shows a stabilizing tool 1908 utilizing one or more balloons 1910 filled with liquid and/or gas. Expansion of the one or more balloons 1910 into lumen 106 may secure working tool 120 within lumen 106. In the embodiment shown in FIG. 19, balloon 1910 secures working tool 120 against a periphery of lumen 106. However, it is contemplated that balloon 1910 may be ring-shaped and extend around lumen 106. In this embodiment, expansion of balloon 1910 may secure working tool 120 at a center (or otherwise at an interior) of lumen 106, as opposed to at its periphery. Stabilizing tool 1908 may be integrated into a cap 1906 configured to attach to distal end 102 of scope 100. Balloon 1910 may be adapted as part of cap 1906. In some embodiments, balloon 1910 is not positioned entirely inside the lumen 106, but rather is partially disposed in or blocks a portion of the distal exit of lumen 106. Stabilizing tool 1908 may include a single (exactly one) balloon 1910 or multiple balloons 1910. Balloon 1910 may be filled using lens wash or insufflation of the scope 100, and balloon 1910 may be coupled to a pressure relief valve (not shown) to help achieve a desired fill level or pressure of the filling fluid (e.g., lens wash or insufflation). Once the balloon 1910 has been filled to a sufficient amount, the pressure relief valve may divert excess fluid out of scope 100 instead of into the one or more balloons 1910. In scopes 100 with forward water jet capability, a forward water jet 1920 (disposed in scope 100) may be used to fill the balloon 1910. When the forward water jet 1920 is activated by the user, a portion of the water may be deflected through a channel 1922 (positioned distal to the distal face of scope 100), and into the displacement balloon 1910 positioned within lumen 106. The balloon material may be flexible (e.g., latex). The balloon 1910 could be shaped in a variety of different ways (e.g., circular, cylindrical, or another suitable shape) to achieve desired placement within lumen 106. Balloon 1910 may be deflated by applying suction to channel 1922. Balloon 1910 also may also deflate due to its own elasticity. For example, upon engaging forward water jet 1920, a portion of the stream may be diverted to inflate balloon 1910. Once forward water jet 1920 has been stopped, balloon 1910 may return to its original, deflated state.

The balloon(s) 1910 also may be filled with an external supply of liquid (e.g., filling using a syringe) or gas (e.g., compressed gas with pressure/flow regulator). Alternatively, the interior of balloon 1910 also may include an electroactive polymer. Passing an electrical current through the electroactive polymer may cause the polymer chains to expand and subsequently inflate the balloon. Substituting the fluidics tubing for electrical wiring may be advantageous for managing space constraints at the distal end 102 of the scope 100.

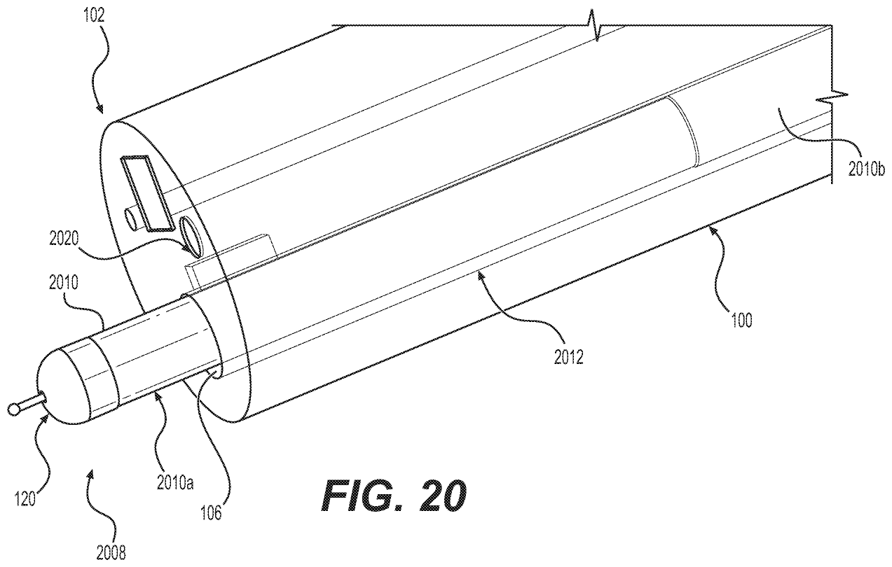

FIGS. 20-22 show embodiments that incorporate a magnetic system for the positioning of working tool 120 within lumen 106. A combination of ferromagnetic materials and magnets may be used to appropriately position working devices 120 within lumen 106.

Referring to the embodiment of FIG. 20, a stabilizing device 2008 may include a first sleeve 2010 that is movable through lumen 106. First sleeve 2010 may include a lumen (not shown) configured to receive working tool 120. First sleeve 2010 may be fixed to working tool 120, or may be manufactured as part of working tool 120. First sleeve 2010 may include one or more areas having magnetic material (e.g., distal portion 2010a) and one or more areas that are non-magnetic (e.g., proximal portion 2010b). When first sleeve 2010 is within range or otherwise adjacent to magnetic material of a second sleeve 2012, the magnetic attraction may help secure working tool 120 throughout the duration of a procedure. Second sleeve 2012 may be inserted into lumen 106 and may include magnetic material at its distal end. Alternatively, second sleeve 2012 may be integral with scope 100, and may comprise the portions of scope 100 that define lumen 106. The strength of the magnet(s) may determine the forces of the adherence and may reflect the force needs of the procedure. To remove working tool 120, the magnetic field can be broken by pulling first sleeve 2010 (and/or working tool 120) and sheering the magnetic field. The magnetic system may include two magnets (both first sleeve 2010 and second sleeve 2012), or one magnet and one ferromagnetic material (either a ferromagnetic second sleeve 2012 and magnetic first sleeve 2010, or a magnetic second sleeve 2012, and ferromagnetic first sleeve 2010) near distal end 102 of the scope 100. In one embodiment, a magnet 2020 is disposed in or adjacent second sleeve 2012, and first sleeve 2010 may be ferromagnetic at its distal end. In some embodiments, the magnetic or ferromagnetic second sleeve 2012 could be integrated into a cap (e.g., cap 800) placed over the distal end 102 of scope 100, and inserted directly into lumen 106. The magnetic or ferromagnetic first sleeve 2010 may be single-use and optimized to securely hold working tool 120. In some embodiments, first sleeve 2010 and/or second sleeve 2012 may include a ferromagnetic-infused polymer shaft.

Stabilizing tool 2008 could position working tool 120 in a variety of different locations within lumen 106 (e.g., a center of lumen 106 or along its edge). The precise positioning of tool 120 may be tuned for preferred visualization and accessory placement at distal end 102.

The magnetic field of the magnet(s) may either be generated using rare earth alloys (e.g., Neodymium) or electric current (electromagnet). The magnet(s) of first sleeve 2010 and/or second sleeve 2012 may have a variety of different shapes including, but not limited to ring, bar, and disk. First sleeve 2010 and/or second sleeve 2012 may be ferromagnetic around their respective entire circumferences, or may be ferromagnetic or magnetic only at discrete points, such that a remainder of the sleeve circumference may be comprised of a nonmagnetic material or polymer. By doing this, working tool 120 can be placed at the periphery of lumen 106, closest to the magnetic material.

The magnetic system may be either passive or active. In the case of passive magnetic systems, positioning of the working tool 120 may be achieved without intervention once working tool 120 is inserted into scope 100. In a passive system, once working tool 120 is secured by the magnetic attractions discussed above, the user may only be able to manipulate working tool 120 by manipulating scope 100, or by removing working tool 120 from the scope entirely. However, in the case of active magnetic systems, the positioning of working tool 120 may require additional intervention from the user. Active magnetic systems, for example, may have direction control that enables the user (e.g., physician) to steer (e.g., rotate) working tool 120 about lumen 106 at distal end 102 (e.g., in a manner similar to the elevator action in a duodenoscope). Direction control for the active magnetic system may be achieved via mechanical power (e.g. twisting of the working tool 120 or a knob by the user) or electrical power (e.g., a servomotor).

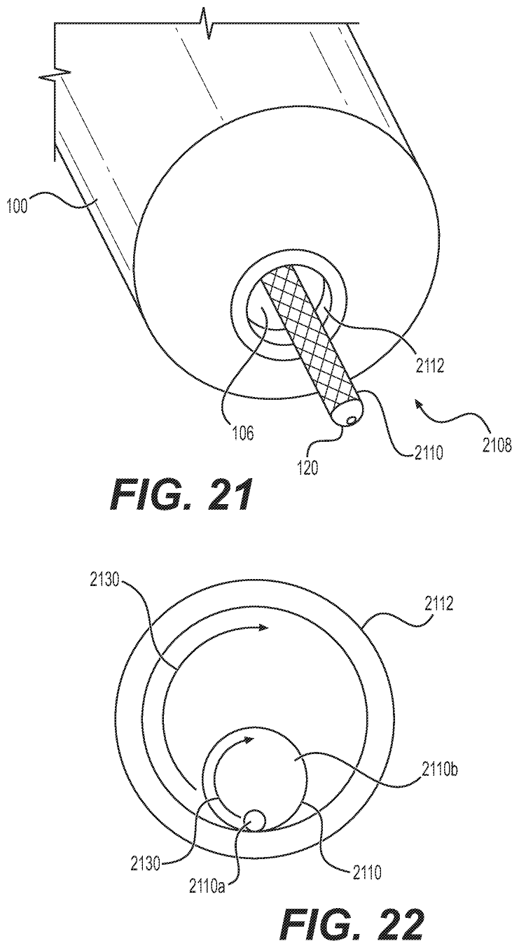

An active magnetic stabilizing tool 2108 is shown in FIGS. 21 and 22. Stabilizing tool 2108 may include a partially ferromagnetic first sleeve 2110 and a magnetic second sleeve 2112. Second sleeve 2112 may include a ring magnet, and may be designed for single-use. A portion of first sleeve 2110 may be ferromagnetic or magnetic (2110a), while another portion may be non-magnetic (2110b). The magnetic attraction force between the ring magnet of second sleeve 2112 and the ferromagnetic portion of first sleeve 2110 firmly holds working tool 120 to the wall surrounding lumen 106. The embodiment of FIGS. 21 and 22 also may allow for directional control by the user. Rotation of the first sleeve 2110 in a direction 2130 (e.g., clockwise) may move first sleeve 2110 around the inner edge of the ring magnet within second sleeve 2112 in the same direction 2130 (e.g., clockwise), as the ferromagnetic material continually loses and regains contact with the ring magnet. It also may give the physician tactile confirmation along with the visual feedback that they have reached the distal end 102 with working tool 120. First sleeve 2110 may be magnetic/ferromagnetic along only a portion of its circumference. For example, magnetic/ferromagnetic material may extend around, e.g., 5 to 95 percent of the circumference of first sleeve 2010, to facilitate sheering of the magnetic field when first sleeve 2110 is rotated. However, it is also contemplated that at least some portions of first sleeve 2010 are magnetic/ferromagnetic around an entirety of its circumference.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and embodiments be considered as exemplary only.

We claim:

1. A system, comprising:
a member having a lumen; and
a stabilizer positioned at a distal end of the lumen, wherein:
the stabilizer includes at least two radially-inward projections circumferentially spaced apart from one another at the distal end of the lumen;
the at least two radially-inward projections defining an opening at the distal end of the lumen; and
a cross-sectional area of the opening is less than a cross-sectional area of the lumen.

2. The system of claim 1, wherein each of the at least two projections is a ramp that extends from a proximal end toward a distal end, and has an increasing radial dimension extending from the proximal end toward the distal end.

3. The system of claim 2, wherein the stabilizer includes a ring-shaped support secured within the lumen, and each of the ramps extends into the lumen from the ring-shaped support.

4. The system of claim 1, wherein the stabilizer includes a cap configured to extend over a distal end of the scope.

5. The system of claim 2, wherein:
the stabilizer includes a first ring positioned within the lumen, and a second ring disposed within the first ring, and rotatable relative to the first ring; and
the ramps extend into the lumen from the second ring.

6. The system of claim 5, further including a working tool insertable into the lumen.

7. The system of claim 6, wherein:
the first ring includes a circumferential flange; and
the ramps are configured to urge the working tool against the circumferential flange.

8. The system of claim 7, wherein rotation of the working tool in a first direction causes the working tool to rotate along the circumferential flange and around the lumen in a second direction that is opposite of the first direction.

9. The system of claim 8, wherein rotation of the working tool in the first direction also causes the second ring to rotate in the second direction.

10. The system of claim 8, wherein:
when the first direction is clockwise, the second direction is counter-clockwise; and
when the first direction is counter-clockwise, the second direction is clockwise.

11. The system of claim 2, wherein the ramps are configured to rotate about a central longitudinal axis of the lumen.

12. The system of claim 11, wherein the stabilizing tool includes one or more gears configured to rotate the ramps.

13. The system of claim 12, further including a twistable member extending from a proximal end of the member to one of the gears, wherein rotation of the twistable member is configured to rotate each of the gears and the ramps.

14. The system of claim 1, wherein a free end of each of the radially-inward projections is configured to flex distally away from a distal end of the member.

15. The system of claim 14, further including a working tool insertable into the lumen, wherein:
the radially-inward projections extend into the lumen from a first side of the lumen;
are configured to be flexed distally by the working tool; and
bias the working tool toward a second side of the lumen that is across a central longitudinal axis of the lumen from the first side.

16. A system, comprising:
a member having a lumen; and
a flexible stabilizer fixed to a distal end of the lumen, wherein:
the flexible stabilizer is movable from a collapsed position to an expanded position extending distally away from the distal end of the lumen;
the flexible stabilizer includes a central longitudinal axis; and
the flexible stabilizer is biased toward the collapsed position and toward a central longitudinal axis of the flexible stabilizer, to define an instrument-containing space having a cross-sectional dimension less than a diameter of the lumen.

17. The system of claim 16, wherein the flexible stabilizer is a coil, spring, or ribbon, and the lumen includes a central longitudinal axis that is offset from the central longitudinal axis of the coil, the spring, or the ribbon.

18. A system, comprising:
a member having a lumen; and
a first sleeve dimensioned to be received in the lumen; and
a first magnet disposed within or adjacent to a distal end of the lumen, wherein:
a distal end of the first sleeve includes a second magnet or a ferromagnetic material;
a proximal end of the first sleeve is non-magnetic; and
the first sleeve includes a lumen configured to receive a working tool.

19. The system of claim 18, wherein the first magnet is a ring surrounding the lumen.

20. The system of claim 19, wherein the second magnet or the ferromagnetic material of the first sleeve extends only partially around a circumference of the first sleeve, and rotation of the first sleeve in a first direction causes the first sleeve to rotate around the lumen in the first direction.
